(19) United States
(12) Patent Application Publication
(10) Pub. No.: US 2026/0145179 A1
(43) Pub. Date: May 28, 2026

(54) BIS-LIGAND CHALCOPYRITE COLLECTOR AND PREPARATION METHOD AND APPLICATION THEREOF

(71) Applicant: Qingdao University of Science and Technology, Qingdao City (CN)

(72) Inventors: Xingrong Zhang, Qingdao City (CN); Xiaokun Fu, Qingdao City (CN); Lulin Lu, Qingdao City (CN); Xinyao Jiang, Qingdao City (CN); Cong Peng, Qingdao City (CN); Yao Meng, Qingdao City (CN); Haoran Cheng, Qingdao City (CN); Ruiyang Zhao, Qingdao City (CN); Hongru Zhang, Qingdao City (CN)

(21) Appl. No.: 19/454,268

(22) Filed: Jan. 20, 2026

(30) Foreign Application Priority Data
Mar. 26, 2025 (CN) 202510360858.7

(51) Int. Cl. B03D 1/012; C07C 333/04; C07C 335/08

(52) U.S. Cl. CPC B03D 1/012; C07C 333/04; C07C 335/08; B03D 2203/02

(57) ABSTRACT

A novel bis-ligand chalcopyrite collector and a preparation method and application thereof. The collector is prepared by first synthesizing an intermediate oxalyl diisothiocyanate from oxalyl chloride and thiocyanate, and then reacting the intermediate with an amine or an alcohol using a one-pot method. The collector provided by the present disclosure can form more stable dual-site adsorption on the chalcopyrite surface, thereby generating a more stable double-chelate-ring structure than the single-site adsorption of a traditional chalcopyrite collector such as xanthate and dithiophosphate. The collector not only has higher collection ability and excellent selectivity, but also can achieve efficient copper-sulfur separation even under a low-alkalinity condition. The preparation process of the collector is simple and operable, facilitating the flotation of chalcopyrite and its separation from pyrite.

### TECHNICAL FIELD

The present disclosure belongs to the technical field of copper-sulfur ore mineral flotation, and specifically relates to a bis-ligand chalcopyrite collector and a preparation method and application thereof.

### BACKGROUND

Copper is widely regarded as a modern energy metal and is one of the most widely used metals worldwide. In copper sulfide ores, copper mainly originates from chalcopyrite, which is usually associated with pyrite. Therefore, effective separation of chalcopyrite from pyrite is essential to obtain qualified copper concentrates.

Meanwhile, due to overexploitation of sulfide ores, grades of copper sulfide ores have become increasingly low. Therefore, flotation is widely used to produce high-grade copper concentrates and to separate valuable minerals from non-target minerals during beneficiation.

As is well known, collectors are crucial in copper-sulfur flotation. They can adsorb onto the surface of a target mineral through chemisorption, Van der Waals forces, and related interfacial forces, thereby rendering the target mineral surface hydrophobic. However, traditional collectors often show limited selectivity and collecting ability because their interaction with mineral surfaces is typically dominated by a single functional group, resulting in a single-site adsorption. For example, xanthates, as the traditional collectors, exhibit poor selectivity for chalcopyrite and generally achieve selective collection only under strongly alkaline conditions. In contrast, Z-200, a xanthate derivative, is widely used in industry due to its high selectivity under relatively low-alkalinity conditions, but its collection ability is lower than that of xanthate.

Accordingly, the present disclosure aims to provide a chalcopyrite collector capable of enhancing the adsorption capacity via a double-bond functional group, thereby achieving high collecting ability and high selectivity under low-alkalinity conditions.

### SUMMARY

In view of the above problems, the present disclosure provides a bis-ligand chalcopyrite collector, and a preparation method and application thereof. The collector is an oxalyl bis-ligand chalcopyrite collector that can achieve flotation separation of chalcopyrite and pyrite under low-alkalinity conditions at a low dosage.

The present disclosure is implemented by the following technical solutions.

Provided is a novel bis-ligand chalcopyrite collector, which is a compound with a structure shown in formula (1) or formula (2):

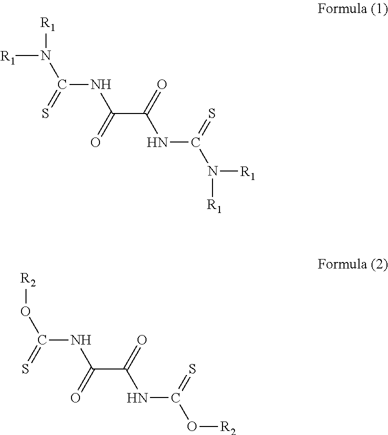

wherein R₁ in formula (1) is a C1-C6 alkyl or aryl, and R₂ in formula (2) is a C1-C7 alkyl or aryl.

Further, R₁ is selected from methyl, ethyl, propyl, isopropyl, butyl, isobutyl, pentyl, isopentyl, phenyl and cyclohexyl; and R₂ is any one of methyl, ethyl, propyl, isopropyl, butyl, isobutyl, pentyl, isopentyl, phenyl, cyclohexyl and benzyl.

Further, the preparation method includes:
(1) using tetrahydrofuran (THF) as a solvent and using oxalyl chloride and thiocyanate as raw materials, preparing an intermediate oxalyl diisothiocyanate by mixing and stirring at a certain temperature; and
(2) reacting the intermediate oxalyl diisothiocyanate with any one of di-n-butylamine, diethylamine, isopropanol and isobutanol in a one-pot process at a certain temperature to obtain the novel bis-ligand chalcopyrite collector containing two C═S bonds.

Further, in the step (1), a molar ratio of any one of di-n-butylamine, diethylamine, isopropanol and isobutanol to oxalyl chloride to thiocyanate is (2 to 2.1):1:(2.05 to 2.1).

Further, in the step (1), the reaction temperature ranges from 0° C. to 5° C., and the reaction time ranges from 2.5 h to 3.5 h;
in the step (2), when di-n-butylamine or diethylamine reacts with the intermediate oxalyl diisothiocyanate, the reaction temperature ranges from 15° C. to 25° C., and the reaction time ranges from 7 h to 8 h; and
in the step (2), when isobutanol or isopropanol reacts with intermediate oxalyl diisothiocyanate, the reaction temperature ranges from 65° C. to 85° C., and the reaction time ranges from 5 h to 6 h.

Provided is an application of a novel oxalyl bis-ligand chalcopyrite collector, wherein the collector is used for flotation separation of chalcopyrite and pyrite and has a structure shown in formula (1) or formula (2).

Further, the pH of pulp in flotation is controlled at 8.0 to 11.0; and
further, a frother in flotation is methyl isobutyl carbinol (MIBC); and the MIBC is added at 8 mg to 10 mg per liter of pulp.

Further, when the novel bis-ligand chalcopyrite collector with the structure shown in formula (1) is adopted, the collector is added in flotation at 1.5 mg to 2.0 mg per liter of pulp; and
when the novel bis-ligand chalcopyrite collector with the structure shown in formula (2) is adopted, the collector is added in flotation at 0.6 mg to 1.0 mg per liter of pulp.

The present disclosure has the following beneficial technical effects.

(1) The collector provided by the present disclosure contains two C═S bonds that enable bidentate adsorption on chalcopyrite surface, thereby forming a more stable chelate-ring adsorption than a single-site adsorption. As a result, the collecting ability and selectivity for chalcopyrite are enhanced, and the flotation separation of chalcopyrite from pyrite can be achieved under lower-alkalinity conditions.

(2) The collector provided by the present disclosure is prepared by a one-pot method with relatively simple reaction conditions and readily available raw materials, and can serve as an industrial alternative to traditional collectors such as Z-200.

### BRIEF DESCRIPTION OF DRAWINGS

FIG. 1 is an infrared spectrum of BDBAO prepared in an embodiment 1 of the present disclosure;

FIG. 2 is a mass spectrum of BDBAO prepared in an embodiment 1 of the present disclosure;

FIG. 3 is an infrared spectrum of DBOBO prepared in an embodiment 2 of the present disclosure;

FIG. 4 is a mass spectrum of DBOBO prepared in an embodiment 2 of the present disclosure;

FIG. 5 is an infrared spectrum of BDBOO prepared in an embodiment 3 of the present disclosure;

FIG. 6 is a mass spectrum of BDBOO prepared in an embodiment 3 of the present disclosure;

FIG. 7 is an infrared spectrum of DPOBO prepared in an embodiment 4 of the present disclosure;

FIG. 8 is a mass spectrum of DPOBO prepared in an embodiment 4 of the present disclosure; and

FIG. 9 is a flotation flowsheet used in embodiments 2 to 4.

### DETAILED DESCRIPTION OF THE EMBODIMENTS

In order to make the objects, technical solutions and advantages of the present disclosure clearer, the present disclosure will be further described in detail below in conjunction with accompanying drawings and embodiments. It should be understood that specific embodiments described herein are only intended to explain the present disclosure, rather than to limit the present disclosure.

Unless otherwise defined, all technical terms used herein have the same meanings as those commonly understood by those skilled in the art. The technical terms used herein are only for the purpose of describing embodiments, and are not intended to limit the protection scope of the present disclosure.

### Embodiment 1: Preparation of Oxalyl Bis-Ligand Chalcopyrite Collector BDBAO

0.0315 mol of ammonium thiocyanate was dissolved in THF (50 mL), and stirred at 0° C. to 5° C. for 20 min. Separately, 0.015 mol of oxalyl chloride was added into a 250 mL three-necked flask, 25 mL of THF was added, and the solution was stirred at 0° C. to 5° C. for 15 min. The ammonium thiocyanate solution was then added dropwise to the oxalyl chloride solution at 0° C. to 5° C. over about 30 min. After addition, the mixture was maintained at 0° C. to 5° C. for 3 h. The ice-water bath was then removed, and 0.03 mol of di-n-butylamine was added dropwise over about 20 min. After the dropwise addition, the reaction solution reached room temperature and was stirred for 8 h. The reaction was monitored by thin-layer chromatography (TLC) (ethyl acetate:petroleum ether:glacial acetic acid=7.5:2.5:0.2). After completion, the solvent was removed by rotary evaporation to obtain a crude product. The crude product was dissolved in 55 g of ethyl acetate and extracted with 30 g of deionized water three times, followed by extraction with deionized water adjusted to pH 4.0 with hydrochloric acid three times. The organic phase of the ethyl acetate was dried over anhydrous magnesium sulfate, and evaporation was performed under reduced pressure to obtain a target product with a yield of 83%.

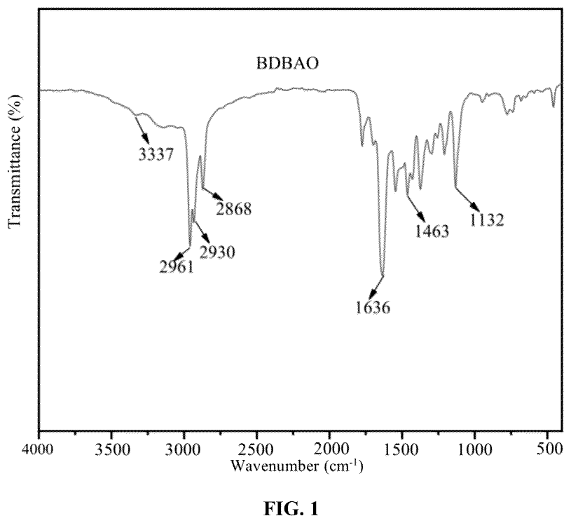

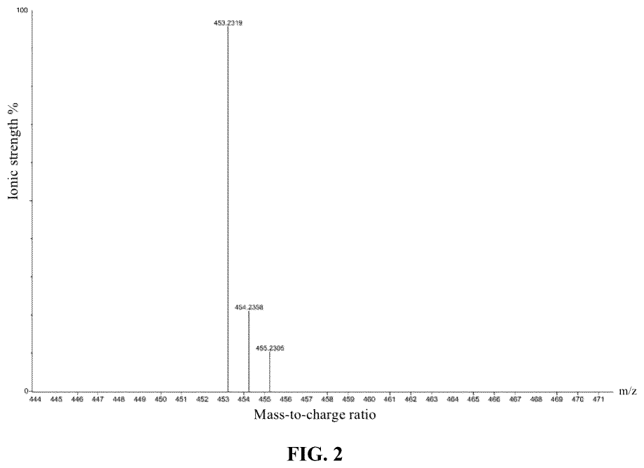

The obtained product was structurally characterized, wherein an infrared spectrum is shown in FIG. 1, a mass spectrum is shown in FIG. 2, and infrared spectrum analysis is shown in Table 1.

TABLE 1
Infrared spectrum analysis of oxalyl bis-ligand collector BDBAO

| Name of compound | Peak displacement and probable attribution | |
|---|---|---|
| N,N′-Oxalyl-N″,N″′- | 3337 | N—H stretching vibration |
| dibutylbis(thiourea) | 2965, 2930, 2868 | CH₃ and CH₂ stretching |
| | | vibration |
| | 1636 | C═O stretching vibration |
| | 1463 | —NH—C(═S)—N stretching |
| | | vibration |
| | 1132 | C═S stretching vibration |

Mass spectrum analysis (EI): 453.2312 [430.2436+Na]⁺.

The collector as a compound prepared in this embodiment 1 is N,N′-Oxalyl-N″,N′″-dibutylbis(thiourea) (BDBAO) with a structure shown as follows:

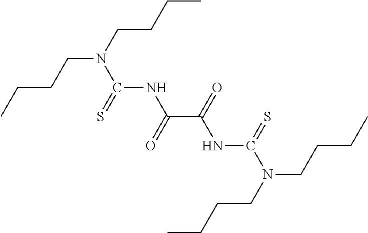 [SMILES: CCCCN(CCCC)C(=S)NC(=O)C(=O)NC(=S)N(CCCC)CCCC]

A preparation and synthesis route of the collector (BDBAO) in this embodiment 1 is shown as:

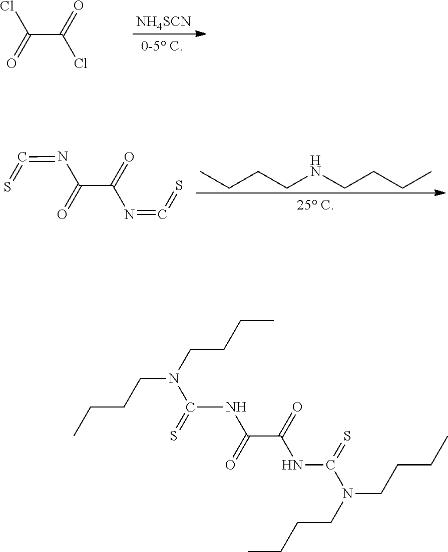

### Embodiment 2: Preparation of Oxalyl Bis-Ligand Collector DBOBO

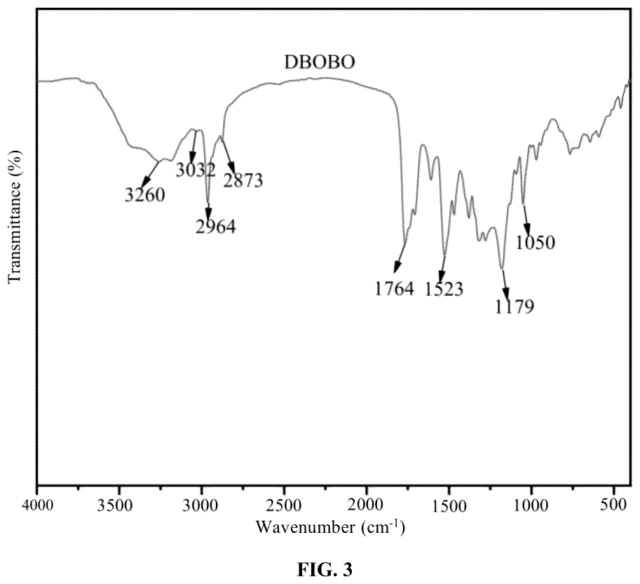

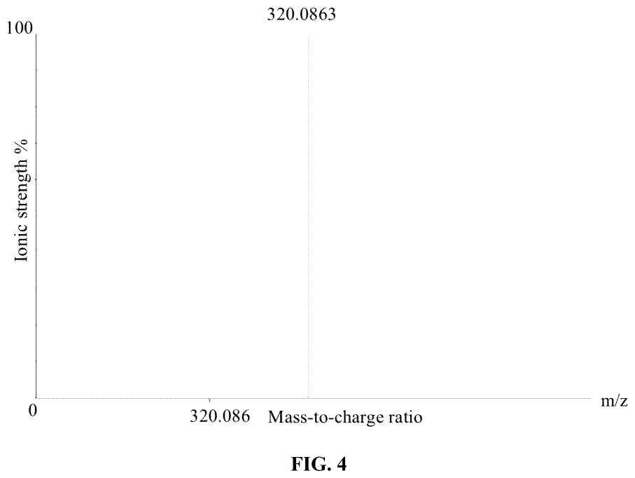

0.0315 mol of ammonium thiocyanate was dissolved in THF (50 mL) and stirred at 0° C. to 5° C. for 20 min. In a 250 mL three-necked flask, 0.015 mol of oxalyl chloride was dissolved in 25 mL of THF and stirred at 0° C. to 5° C. for 15 min. The ammonium thiocyanate solution was slowly added dropwise to the oxalyl chloride solution at 0° C. to 5° C. (for about 30 min). The mixture was maintained at 0° C. to 5° C. for 3 h, and then 0.03 mol of isobutanol was added dropwise (for about 20 min). The reaction mixture was stirred at 75° C. for 6 h and monitored by thin-layer chromatography (TLC) (ethyl acetate:petroleum ether:glacial acetic acid=7.5:2.5:0.2). After completion, the solvent was removed by rotary evaporation to obtain a crude product. The crude product was dissolved in 55 g of ethyl acetate, extracted with 30 g of deionized water three times, and then extracted with deionized water adjusted to pH 4.0 using hydrochloric acid three times. The organic phase of the ethyl acetate was dried over anhydrous magnesium sulfate, and concentrated under reduced pressure to obtain a target product with a yield of 69%. The obtained product was structurally characterized, wherein an infrared spectrum is shown in FIG. 3, a mass spectrum is shown in FIG. 4, and infrared spectroscopic analysis is shown in Table 2.

TABLE 2
Infrared spectroscopic analysis for
oxalyl bis-ligand collector DBOBO

| Name of compound | Peak displacement and probable attribution | |
|---|---|---|
| O,O′-diisobutyl oxalyl | 3260 | N—H stretching vibration |
| biscarbamate | 3032, 2964, 2873 | CH₃ and CH₂ stretching |
| | | vibration |
| | 1764 | C═O stretching vibration |
| | 1523 | H—N—C(═S)—O stretching |
| | | vibration |
| | 1179 | C═S stretching vibration |
| | 1050 | C—O stretching vibration |

Mass spectrum analysis (EI): 320.0883.

The collector as a compound obtained in this embodiment 2 is O,O′-diisobutyl oxalyl biscarbamate (DBOBO) with a structure shown as follows:

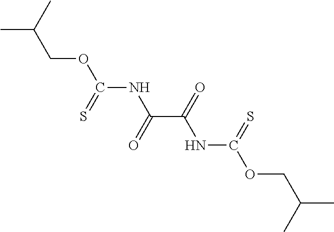 [SMILES: CC(C)COC(=S)NC(=O)C(=O)NC(=S)OCC(C)C]

A preparation and synthesis route of the collector (DBOBO) in this embodiment 2 is shown as:

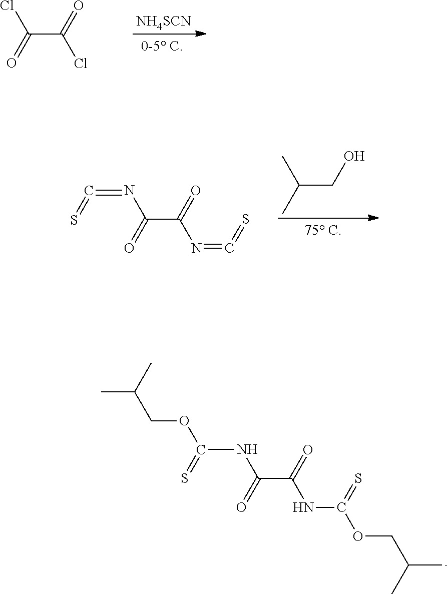

### Embodiment 3: Preparation of Oxalyl Bis-Ligand Collector BDBOO

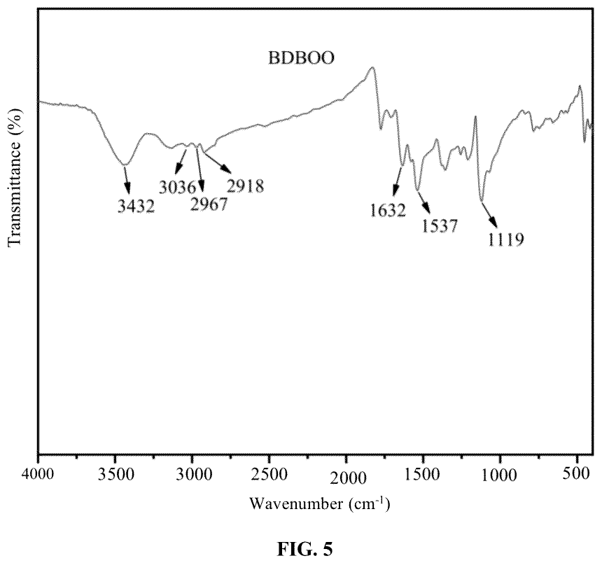

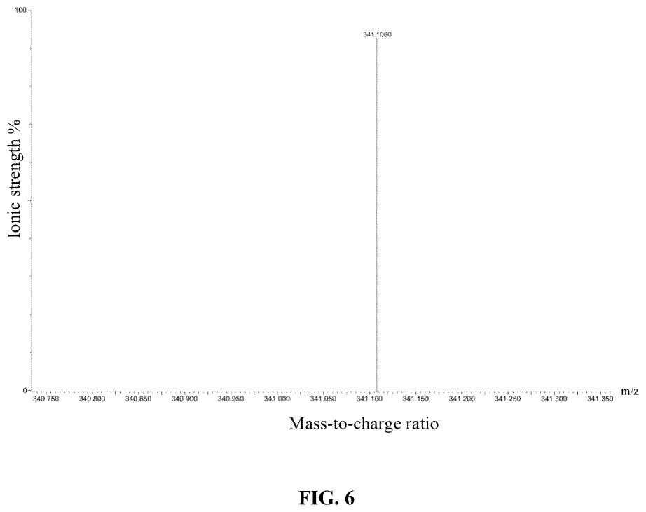

0.0315 mol of ammonium thiocyanate was dissolved in THF (50 mL) and stirred at 0° C. to 5° C. for 20 min. In a 250 mL three-necked flask, 0.015 mol of oxalyl chloride was dissolved in 25 mL of THF and stirred at 0° C. to 5° C. for 15 min. The ammonium thiocyanate solution was slowly added dropwise to the oxalyl chloride solution at 0° C. to 5° C. (for about 30 min). The mixture was maintained at 0° C. to 5° C. for 3 h. After removal of the ice-water bath, 0.03 mol of diethylamine was added dropwise over about 20 min. The reaction solution reached approximately room temperature and was stirred for 8 h, and monitored by thin-layer chromatography (TLC) (ethyl acetate:petroleum ether:glacial acetic acid=7.5:2.5:0.2). After completion, the solvent was removed by rotary evaporation to obtain a crude product. The crude product was dissolved in 55 g of ethyl acetate, extracted with 30 g of deionized water three times, and then extracted with deionized water adjusted to pH 4.0 with hydrochloric acid three times. The organic phase of the ethyl acetate was dried over anhydrous magnesium sulfate and concentrated under reduced pressure to obtain a target product with a yield of 85.1%. The obtained product was structurally characterized, wherein an infrared spectrum is shown in FIG. 5, a mass spectrum is shown in FIG. 6, and infrared spectroscopic analysis is shown in Table 3.

TABLE 3
Infrared spectroscopic analysis for
oxalyl bis-ligand collector BDBOO

| Name of compound | Peak displacement and probable attribution | |
|---|---|---|
| N,N′-Oxalyl-N″,N″′- | 3432 | N—H stretching vibration |
| diethylbis(thiourea) | 3036, 2967, 2918 | CH₃ and CH₂ stretching |
| | | vibration |
| | 1632 | C═O stretching vibration |
| | 1537 | —NH—C(═S)—N stretching |
| | | vibration |
| | 1119 | C═S stretching vibration |

Mass spectrum analysis (EI): 341.1080.

The collector as a compound obtained in this embodiment 3 is N,N′-Oxalyl-N″,N′″-diethylbis(thiourea) (BDBOO) with a structure shown as follows:

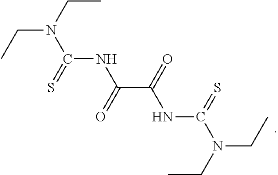 [SMILES: CCN(CC)C(=S)NC(=O)C(=O)NC(=S)N(CC)CC]

### Embodiment 4: Preparation of Oxalyl Bis-Ligand Collector DPOBO

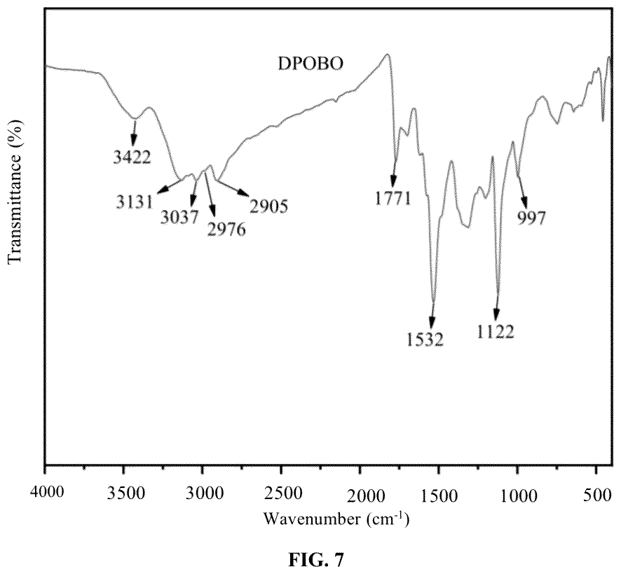

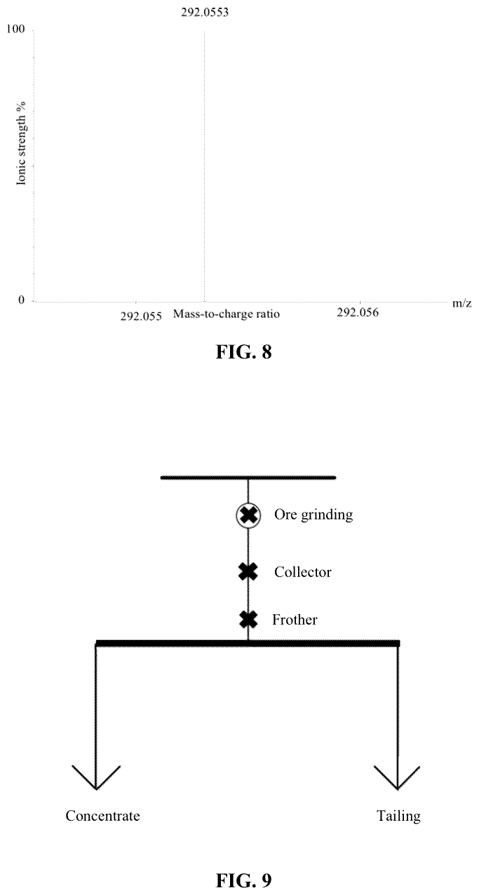

0.0315 mol of ammonium thiocyanate was dissolved in THF (50 mL) and stirred at 0° C. to 5° C. for 20 min. In a 250 mL three-necked flask, 0.015 mol of oxalyl chloride was dissolved in 25 mL of THF and stirred at 0° C. to 5° C. for 15 min. The ammonium thiocyanate solution was slowly added dropwise to the oxalyl chloride solution at 0° C. to 5° C. over about 30 min. The mixture was maintained at 0° C. to 5° C. for 3 h, and then 0.03 mol of isopropanol was added dropwise (for about 20 min). The reaction mixture was stirred at 75° C. for 6 h and monitored by thin-layer chromatography (TLC) (ethyl acetate:petroleum ether:glacial acetic acid=7.5:2.5:0.2). After completion, the solvent was removed by rotary evaporation to obtain a crude product. The crude product was dissolved in 55 g of ethyl acetate, extracted with 30 g of deionized water three times, and then extracted with deionized water adjusted to pH 4.0 with hydrochloric acid three times. The organic phase of the ethyl acetate was dried over anhydrous magnesium sulfate and concentrated under reduced pressure to obtain a target product with a yield of 69%. The obtained product was structurally characterized, wherein an infrared spectrum is shown in FIG. 7, a mass spectrum is shown in FIG. 8, and infrared spectroscopic analysis is shown in Table 4.

TABLE 4
Infrared spectroscopic analysis for
oxalyl bis-ligand collector DBOBO

| Name of compound | Peak displacement and probable attribution | |
|---|---|---|
| O,O′-diisopropyl oxalyl | 3422, 3131 | N—H stretching vibration |
| biscarbamate | 3037, 2976, 2905 | CH₃ and CH₂ stretching |
| | | vibration |
| | 1771 | C═O stretching vibration |
| | 1518 | H—N—C(═S)—O |
| | | stretching vibration |
| | 1178 | C═S stretching vibration |
| | 1048 | C—O stretching vibration |

Mass spectrum analysis (EI): 292.0553.

The collector as a compound obtained in this embodiment 4 is O,O′-diisopropyl oxalyl biscarbamate (DBOBO) with a structure shown as follows:

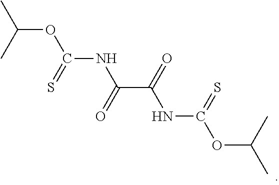 [SMILES: CC(C)OC(=S)NC(=O)C(=O)NC(=S)OC(C)C]

### Embodiment 5: Application of Oxalyl Bis-Ligand Collector to Flotation of Chalcopyrite and Comparison with Traditional Collector Z-200

Flotation was performed on chalcopyrite according to the flotation flowsheet shown in FIG. 9. A 30 mL flotation cell was used, with a fixed impeller speed of 1758 r/min. The pH of pulp was adjusted to 9.0, and MIBC was used as the frother at 10.0 mg/L. Chalcopyrite with a particle size of −400 to −200 mesh was floated for 3 min. The flotation results are shown in the following table.

TABLE 5
Comparison between concentrations of BDBAO, BDBOO, DPOBO,
DBOBO and Z-200 and recoveries of chalcopyrite

| | Concentration | |
|---|---|---|
| Type of | (mg/L) of | Recovery (%) of |
| collector | collector | chalcopyrite |
| Z-200 | 0.6 | 83.26 |
| | 0.8 | 85.37 |
| | 1.5 | 88.26 |
| | 2.0 | 90.6 |
| BDBAO | 1.5 | 95.48 |
| | 2.0 | 96.65 |
| DBOBO | 0.6 | 92.48 |
| | 0.8 | 93.82 |
| BDBOO | 1.5 | 95.91 |
| | 2.0 | 96.32 |
| | 0.6 | 93.52 |
| | 0.8 | 94.73 |

### Embodiment 6: Application of Oxalyl Bis-Ligand Collector to Flotation of Pyrite and Comparison with Traditional Collector Z-200

Flotation was performed on pyrite according to the flotation flowsheet shown in FIG. 9. A 30 mL flotation cell was used, with a fixed impeller speed of 1758 r/min. The pH of pulp was adjusted to 9.0, and MIBC was used as the frother at 10.0 mg/L. Pyrite with a particle size ranging from −400 to −200 mesh was floated for 3 min.

TABLE 6
Comparison between concentrations of BDBAO, BDBOO,
DPOBO, DBOBO and Z-200 and recoveries of pyrite

| | Concentration | |
|---|---|---|
| Type of | (mg/L) of | Recovery (%) of |
| collector | collector | pyrite |
| Z-200 | 0.6 | 1.78 |
| | 0.8 | 2.38 |
| | 1.5 | 3.43 |
| | 2.0 | 5.45 |
| BDBAO | 1.5 | 0.57 |
| | 2.0 | 1.8 |
| DBOBO | 0.6 | 2.06 |
| | 0.8 | 1.21 |
| BDBOO | 1.5 | 1.4 |
| | 2.0 | 2.7 |
| DPOBO | 0.6 | 3.68 |
| | 0.8 | 3.43 |

### Embodiment 7: Application of Oxalyl Bis-Ligand Collector in Flotation Separation of Artificial Mixed Mineral (a Mass Ratio of Chalcopyrite to Pyrite is 1:1) and Comparison with Traditional Collector Z-200

Flotation was performed on the artificial mixed mineral according to the flotation flowsheet shown in FIG. 9. A 30 mL flotation cell was used, with a fixed impeller speed of 1758 r/min. Flotation was performed for 3 min using the bis-ligand collector (2.0 mg/L BDBAO or 0.8 mg/L DBOBO) and MIBC (10.0 mg/L) as the frother at a pH of 9.0 or 10.0 of pulp. For comparison, Z-200 was used as the collector under the same conditions. Concentrates (froth products) and tailings were collected, dried and weighed. Recoveries were calculated according to product weights and the measured element contents, and the results are shown in the following table.

TABLE 7
Effects of concentrations of BDBAO, BDBOO, DPOBO, DBOBO and
Z-200 on flotation separation of the artificial mixed mineral

| | | Grade, % | Recovery, % |
|---|---|---|---|
| | Name of product | Cu | Cu |
| Experimental condition that | | | |
| the pH is 9.0 | | | |
| Collector BDBAO | Concentrate | 26.22 | 90.22 |
| | Tailing | 3.74 | 9.78 |
| Collector Z-200 | Concentrate | 24.50 | 84.16 |
| | Tailing | 6.37 | 15.84 |
| Collector DBOBO | Concentrate | 30.12 | 91.34 |
| | Tailing | 3.67 | 8.66 |
| Collector BDBOO | Concentrate | 28.06 | 91.23 |
| | Tailing | 3.75 | 8.77 |
| Collector DPOBO | Concentrate | 30.26 | 90.22 |
| | Tailing | 3.17 | 9.78 |
| Experimental condition that | | | |
| the pH is 10.0 | | | |
| Collector BDBAO | Concentrate | 28.01 | 89.31 |
| | Tailing | 3.67 | 10.69 |
| Collector Z-200 | Concentrate | 24.30 | 81.55 |
| | Tailing | 6.85 | 18.45 |
| Collector DBOBO | Concentrate | 30.18 | 89.75 |
| | Tailing | 3.12 | 10.25 |
| Collector BDBOO | Concentrate | 28.78 | 92.16 |
| | Tailing | 2.36 | 7.84 |
| Collector DPOBO | Concentrate | 31.14 | 88.25 |
| | Tailing | 4.26 | 11.75 |

The results show that, compared with the traditional collector Z-200, the bis-ligand collector disclosed herein exhibits higher collection ability and excellent selectivity, enabling efficient copper-sulfur separation even under a low-alkalinity condition (pH 9.0). These findings provide theoretical and experimental support for the development of a novel efficient chalcopyrite collector with low-alkalinity applicability and high selectivity. The synthesis and application of N,N′-Oxalyl-N″,N′″-dibutylbis(thiourea) provide a feasible approach to improving the flotation performance of low-grade and complex ores, and are expected to promote advances in copper ore beneficiation technology.

The collector disclosed herein can form more stable dual-site adsorption on the chalcopyrite surface, thereby generating a more stable chelated adsorption structure than the single-site adsorption typically associated with traditional collectors such as xanthates and dithiophosphates. As a result, the collector not only has higher collection ability and excellent selectivity, but also can achieve efficient copper-sulfur separation under a low-alkalinity condition. The preparation process is simple and operable, facilitating chalcopyrite flotation and its separation from pyrite.

The above description is only one of preferred examples of the present disclosure and is not intended to limit the present disclosure. Any modifications, equivalent substitutions, or improvements made within the spirit and principles of the present disclosure should fall within the protection scope of the present disclosure.

What is claimed is:

1. An application of an oxalyl bis-ligand chalcopyrite collector, wherein the bis-ligand chalcopyrite collector is used for flotation separation of chalcopyrite and pyrite and is a compound with a structure shown in formula (1) or formula (2); 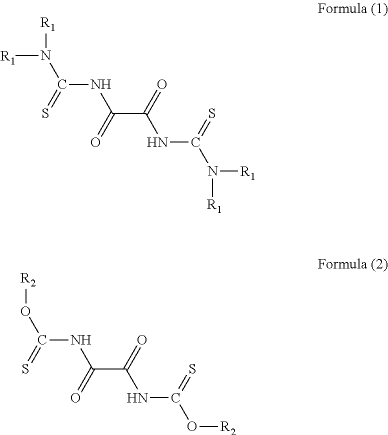
wherein R₁ is any one of methyl, ethyl, propyl, isopropyl, butyl, isobutyl, pentyl, isopentyl and cyclohexyl; and R₂ is any one of methyl, ethyl, propyl, isopropyl, butyl, isobutyl, pentyl, isopentyl and cyclohexyl.

2. The application of the oxalyl bis-ligand chalcopyrite collector according to claim 1, wherein the pH of pulp in flotation is controlled at 8.0 to 11.0.

3. The application of the oxalyl bis-ligand chalcopyrite collector according to claim 2, wherein a frother in flotation is methyl isobutyl carbinol (MIBC); and the concentration of MIBC ranges from 8 mg/L to 10 mg/L.

4. The application of the oxalyl bis-ligand chalcopyrite collector according to claim 3, wherein when the bis-ligand chalcopyrite collector with the structure shown in formula (1) is adopted, the concentration of the collector in flotation ranges from 1.5 mg/L to 2.0 mg/L; and
when the bis-ligand chalcopyrite collector with the structure shown in formula (2) is adopted, the concentration of the collector in flotation ranges from 0.6 mg/L to 1.0 mg/L.

5. The application of the oxalyl bis-ligand chalcopyrite collector according to claim 1, wherein a preparation method of the bis-ligand chalcopyrite collector comprises:
(1) with tetrahydrofuran as a solvent and oxalyl chloride and thiocyanate as raw materials, preparing an intermediate oxalyl diisothiocyanate by mixing and stirring at a certain temperature; and
(2) reacting one of di-n-butylamine, diethylamine, isopropanol and isobutanol with the intermediate oxalyl diisothiocyanate at a certain temperature in a one-pot reaction to prepare the bis-ligand chalcopyrite collector containing two C═S bonds; wherein
in the step (1), the thiocyanate is ammonium thiocyanate or sodium thiocyanate; and
a molar ratio of one of the di-n-butylamine, diethylamine, isopropanol and isobutanol to oxalyl chloride to thiocyanate is (2 to 2.1):1:(2.05 to 2.1).

6. The application of the oxalyl bis-ligand chalcopyrite collector according to claim 5, wherein, in step (1), the reaction temperature ranges from 0° C. to 5° C. and the reaction time ranges from 2.5 h to 3.5 h;
in the step (2), when di-n-butylamine or diethylamine is adopted, the reaction temperature ranges from 15° C. to 25° C. and the reaction time ranges from 7 h to 8 h; and when isobutanol or isopropanol is adopted, the reaction temperature ranges from 65° C. to 85° C. and the reaction time ranges from 5 h to 6 h.
